(19) United States
(12) Patent Application Publication
(10) Pub. No.: US 2012/0022519 A1
(43) Pub. Date: Jan. 26, 2012

(54) SURGICAL CUTTING AND SEALING INSTRUMENT WITH CONTROLLED ENERGY DELIVERY

(75) Inventors: Zhifan F. Huang, Mason, OH (US); David C. Yates, West Chester, OH (US); Gavin M. Monson, Oxford, OH (US)

(73) Assignee: Ethicon Endo-Surgery, Inc., Cincinnati, OH (US)

(21) Appl. No.: 12/841,370

(22) Filed: Jul. 22, 2010

(51) Int. Cl. A61B 18/18; A61B 18/14

(52) U.S. Cl. 606/33; 606/41

(57) ABSTRACT

A surgical instrument for supplying energy to tissue can comprise a jaw member comprising an electrode, wherein the electrode is configured to supply energy from a power source to captured tissue. A control unit is configured to regulate the amount of energy delivered to the tissue through the monitoring of the current flowing through the tissue and the rate of change of the current flowing through the tissue. Through the monitoring of the current, the impedance of the tissue and the rate of change of impedance of the tissue may be determined to monitor the state of the captured tissue and reduce excess tissue heating.

### BACKGROUND

The present invention relates to medical devices and methods. More particularly, the present invention relates to electrosurgical instruments and methods for sealing and transecting tissue.

In various circumstances, a surgical instrument can be configured to apply energy to tissue in order to treat and/or destroy the tissue. In certain circumstances, a surgical instrument can comprise one or more electrodes which can be positioned against and/or positioned relative to the tissue such that electrical current can flow from one electrode, through the tissue, and to the other electrode. The surgical instrument can comprise an electrical input, a supply conductor electrically coupled with the electrodes, and/or a return conductor which can be configured to allow current to flow from the electrical input, through the supply conductor, through the electrodes and the tissue, and then through the return conductor to an electrical output, for example. In various circumstances, heat can be generated by the current flowing through the tissue, wherein the heat can cause one or more hemostatic seals to form within the tissue and/or between tissues. Such embodiments may be particularly useful for sealing blood vessels, for example. The surgical instrument can also comprise a cutting member that can be moved relative to the tissue and the electrodes in order to transect the tissue.

By way of example, energy applied by a surgical instrument may be in the form of radio frequency (“Rf”) energy. Rf energy is a form of electrical energy that may be in the frequency range of 300 kilohertz (kHz) to 1 megahertz (MHz). In application, Rf surgical instruments transmit low frequency radio waves through electrodes, which cause ionic agitation, or friction, increasing the temperature of the tissue. Since a sharp boundary is created between the affected tissue and that surrounding it, surgeons can operate with a high level of precision and control, without much sacrifice to the adjacent normal tissue. The low operating temperatures of Rf energy enables surgeons to remove, shrink or sculpt soft tissue while simultaneously sealing blood vessels. Rf energy works particularly well on connective tissue, which is primarily comprised of collagen and shrinks when contacted by heat.

In various open, endoscopic, and/or laparoscopic surgeries, for example, it may be necessary to coagulate, seal, and/or fuse tissue. One means of sealing tissue relies upon the application of electrical energy to tissue captured within an end effector of a surgical instrument in order to cause thermal effects within the tissue. Various mono-polar and bi-polar radio frequency (Rf) surgical instruments and surgical techniques have been developed for such purposes. In general, the delivery of Rf energy to the captured tissue elevates the temperature of the tissue and, as a result, the energy can at least partially denature proteins within the tissue. Such proteins, such as collagen, for example, may be denatured into a proteinaceous amalgam that intermixes and fuses, or “welds”, together as the proteins renature. As the treated region heals over time, this biological “weld” may be reabsorbed by the body's wound healing process.

In certain arrangements of a bi-polar radio frequency (Rf) surgical instrument, the surgical instrument can comprise opposing first and second jaws, wherein the face of each jaw can comprise an electrode. In use, the tissue can be captured between the jaw faces such that electrical current can flow between the electrodes in the opposing jaws and through the tissue positioned therebetween. Such instruments may have to seal or “weld” many types of tissues, such as anatomic structures having walls with irregular or thick fibrous content, bundles of disparate anatomic structures, substantially thick anatomic structures, and/or tissues with thick fascia layers such as large diameter blood vessels, for example. With particular regard to sealing large diameter blood vessels, for example, such applications may require a high strength tissue weld immediately post-treatment.

The foregoing discussion is intended only to illustrate various aspects of the related art in the field of the invention at the time, and should not be taken as a disavowal of claim scope.

### SUMMARY

In one embodiment, an electrosurgical system may comprise a control unit comprising a processor and memory in communication with the processor, wherein the control unit is configured to deliver a variable level of energy to a circuit comprising an electrode and tissue in electrical communication with the electrode. The control unit may be configured to vary the level of energy delivered to the tissue based on a current flowing through the tissue and a rate of change of the current flowing through the tissue.

In another embodiment, an electrosurgical system may comprise a control unit configured to deliver a variable level of radio frequency (Rf) energy to a circuit comprising an electrode and tissue in electrical communication with the electrode. The control unit may be configured to vary the level of Rf energy delivered to the tissue based on an impedance of the tissue and a rate of change of the impedance of the tissue. The electrosurgical system may further comprise a handle, an elongate shaft extending distally from the handle, and a trigger operably connected to the elongate shaft. The electrosurgical system may also comprise an end effector coupled to the distal end of the elongate shaft, where the end effector comprises a first jaw member and a second jaw member. The first jaw member is moveable relative to the second jaw member between an open and a closed position. The end effector may also comprise an axially movable cutting member.

In yet another embodiment, an apparatus may comprise an energy delivery source configured to electrically couple to a circuit comprising an electrode and tissue. The energy delivery source may comprise a current sensing circuit configured to sense the current through the tissue during the delivery of the energy and a control unit configured to receive a signal from the current sensing circuit and vary an output level of the energy based on an impedance of the tissue and a rate of change of the impedance of the tissue.

The foregoing discussion should not be taken as a disavowal of claim scope.

### FIGURES

Various features of the embodiments described herein are set forth with particularity in the appended claims. The various embodiments, however, both as to organization and methods of operation, together with advantages thereof, may be understood in accordance with the following description taken in conjunction with the accompanying drawings as follows.

FIG. 1 is a perspective view of a surgical instrument according to a non-limiting embodiment.

FIG. 2 is a side view of a handle of the surgical instrument of FIG. 1 with a half of a handle body removed to illustrate some of the components therein.

FIG. 3 is a perspective view of an end effector of the surgical instrument of FIG. 1 illustrated in an open configuration; the distal end of a cutting member is illustrated in a retracted position.

FIG. 4 is a perspective view of the end effector of the surgical instrument of FIG. 1 illustrated in a closed configuration; the distal end of the cutting member is illustrated in a partially advanced position.

FIG. 5 is a perspective sectional view of a portion of a cutting member of the surgical instrument of FIG. 1; the cutting member is shown at least partially shaped like an I-beam.

FIG. 6 is a sectional view of the end effector of FIG. 1

FIG. 7 is a simplified circuit diagram of the surgical instrument during use in accordance with a non-limiting embodiment.

FIG. 8 is a graph of a tissue impedance curve and a PTC material impedance curve as a function of temperature in accordance with a non-limiting embodiment.

FIGS. 9 and 10 are graphs of power level curves over time in accordance with non-limiting embodiments.

Corresponding reference characters indicate corresponding parts throughout the several views. The exemplifications set out herein illustrate various embodiments of the invention, in one form, and such exemplifications are not to be construed as limiting the scope of the invention in any manner.

### DETAILED DESCRIPTION

Various embodiments are directed to apparatuses, systems, and methods for the treatment of tissue. Numerous specific details are set forth to provide a thorough understanding of the overall structure, function, manufacture, and use of the embodiments as described in the specification and illustrated in the accompanying drawings. It will be understood by those skilled in the art, however, that the embodiments may be practiced without such specific details. In other instances, well-known operations, components, and elements have not been described in detail so as not to obscure the embodiments described in the specification. Those of ordinary skill in the art will understand that the embodiments described and illustrated herein are non-limiting examples, and thus it can be appreciated that the specific structural and functional details disclosed herein may be representative and illustrative. Variations and changes thereto may be made without departing from the scope of the claims.

Reference throughout the specification to “various embodiments,” “some embodiments,” “one embodiment,” or “an embodiment”, or the like, means that a particular feature, structure, or characteristic described in connection with the embodiment is included in at least one embodiment. Thus, appearances of the phrases “in various embodiments,” “in some embodiments,” “in one embodiment,” or “in an embodiment”, or the like, in places throughout the specification are not necessarily all referring to the same embodiment. Furthermore, the particular features, structures, or characteristics may be combined in any suitable manner in one or more embodiments. Thus, the particular features, structures, or characteristics illustrated or described in connection with one embodiment may be combined, in whole or in part, with the features structures, or characteristics of one or more other embodiments without limitation.

The entire disclosures of the following non-provisional United States patents are hereby incorporated by reference herein:
U.S. Pat. No. 7,381,209, entitled ELECTROSURGICAL INSTRUMENT;
U.S. Pat. No. 7,354,440, entitled ELECTROSURGICAL INSTRUMENT AND METHOD OF USE;
U.S. Pat. No. 7,311,709, entitled ELECTROSURGICAL INSTRUMENT AND METHOD OF USE;
U.S. Pat. No. 7,309,849, entitled POLYMER COMPOSITIONS EXHIBITING A PTC PROPERTY AND METHODS OF FABRICATION;
U.S. Pat. No. 7,220,951, entitled SURGICAL SEALING SURFACES AND METHODS OF USE;
U.S. Pat. No. 7,189,233, entitled ELECTROSURGICAL INSTRUMENT;
U.S. Pat. No. 7,186,253, entitled ELECTROSURGICAL JAW STRUCTURE FOR CONTROLLED ENERGY DELIVERY;
U.S. Pat. No. 7,169,146, entitled ELECTROSURGICAL PROBE AND METHOD OF USE;
U.S. Pat. No. 7,125,409, entitled ELECTROSURGICAL WORKING END FOR CONTROLLED ENERGY DELIVERY; and
U.S. Pat. No. 7,112,201, entitled ELECTROSURGICAL INSTRUMENT AND METHOD OF USE.

Various embodiments of systems and methods of the invention relate to creating thermal “welds” or “fusion” within native tissue volumes. The alternative terms of tissue “welding” and tissue “fusion” may be used interchangeably herein to describe thermal treatments of a targeted tissue volume that result in a substantially uniform fused-together tissue mass, for example, in welding blood vessels that exhibit substantial burst strength immediately post-treatment. The strength of such welds is particularly useful for (i) permanently sealing blood vessels in vessel transection procedures; (ii) welding organ margins in resection procedures; (iii) welding other anatomic ducts wherein permanent closure is required; and also (iv) for performing vessel anastomosis, vessel closure or other procedures that join together anatomic structures or portions thereof. The welding or fusion of tissue as disclosed herein is to be distinguished from “coagulation”, “hemostasis” and other similar descriptive terms that generally relate to the collapse and occlusion of blood flow within small blood vessels or vascularized tissue. For example, any surface application of thermal energy can cause coagulation or hemostasis—but does not fall into the category of “welding” as the term is used herein. Such surface coagulation does not create a weld that provides any substantial strength in the treated tissue.

At the molecular level, the phenomena of truly “welding” tissue as disclosed herein may result from the thermally-induced denaturation of collagen and other protein molecules in a targeted tissue volume to create a transient liquid or gel-like proteinaceous amalgam. A selected energy density is provided in the targeted tissue to cause hydrothermal breakdown of intra- and intermolecular hydrogen crosslinks in collagen and other proteins. The denatured amalgam is maintained at a selected level of hydration—without desiccation—for a selected time interval which can be very brief. The targeted tissue volume is maintained under a selected very high level of mechanical compression to insure that the unwound strands of the denatured proteins are in close proximity to allow their intertwining and entanglement. Upon thermal relaxation, the intermixed amalgam results in protein entanglement as re-crosslinking or renaturation occurs to thereby cause a uniform fused-together mass.

It will be appreciated that the terms “proximal” and “distal” may be used throughout the specification with reference to a clinician manipulating one end of an instrument used to treat a patient. The term “proximal” refers to the portion of the instrument closest to the clinician and the term “distal” refers to the portion located furthest from the clinician. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

Various embodiments disclosed herein provide electrosurgical jaw structures adapted for transecting captured tissue between the jaws and for contemporaneously welding the captured tissue margins with controlled application of Rf energy. The jaw structures may comprise a scoring element which may cut or score tissue independently of the tissue capturing and welding functions of the jaw structures. The jaw structures may comprise first and second opposing jaws that carry positive temperature coefficient (PTC) bodies for modulating Rf energy delivery to the engaged tissue.

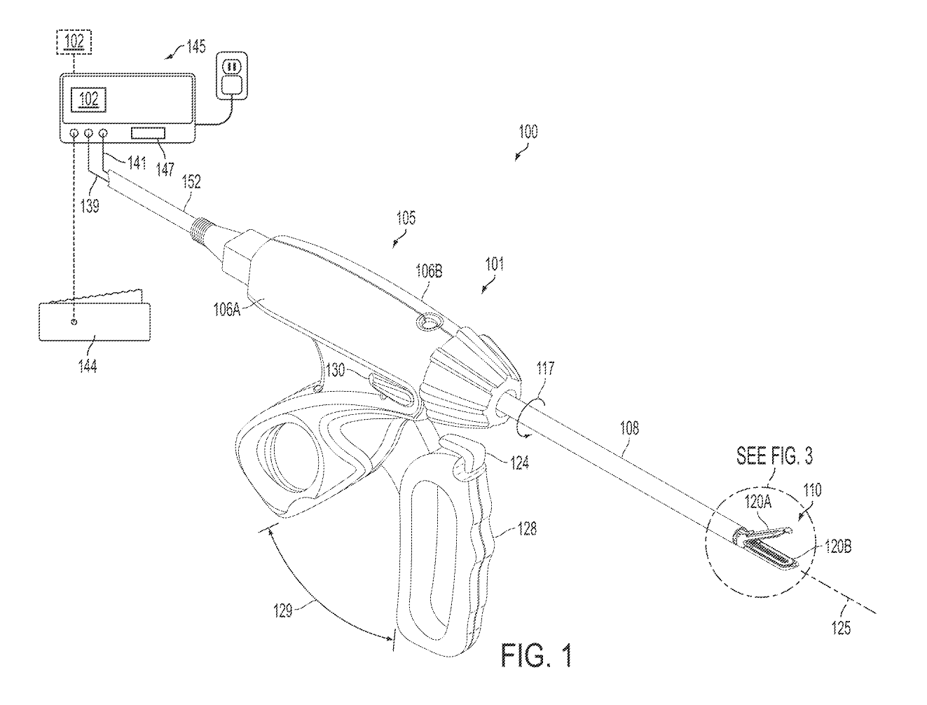

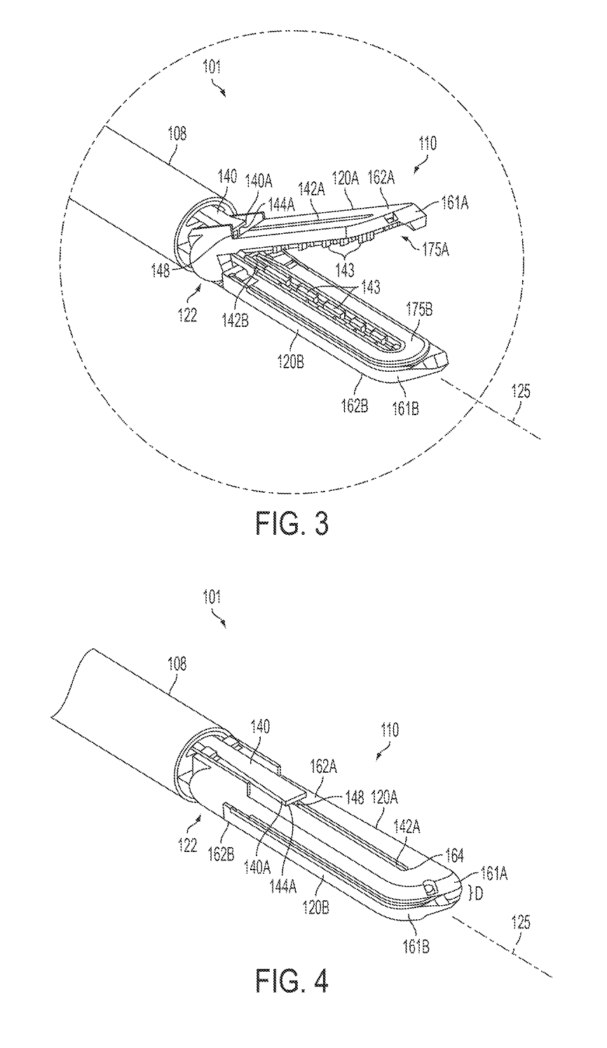

A surgical instrument can be configured to supply energy, such as electrical energy and/or heat energy, to the tissue of a patient. For example, various embodiments disclosed herein provide electrosurgical jaw structures adapted for transecting captured tissue between the jaws and for contemporaneously welding the captured tissue margins with controlled application of Rf energy. Referring now to FIG. 1, an electrosurgical system 100 is shown in accordance with various embodiments. The electrosurgical system 100 includes an electrosurgical instrument 101 that may comprise a proximal handle 105, a distal working end or end effector 110 and an introducer or elongate shaft 108 disposed in-between. The end effector 110 may comprise a set of openable-closeable jaws with straight or curved jaws—an upper first jaw 120A and a lower second jaw 120B. The first jaw 120A and the second jaw 120B may each comprise an elongate slot or channel 142A and 142B (see FIG. 3), respectively, disposed outwardly along their respective middle portions.

The electrosurgical system 100 includes a generator 145 in electrical communication with the electrosurgical instrument 101. The generator 145 is connected to electrosurgical instrument 101 via a suitable transmission medium such as a cable 152. In one embodiment, the generator 145 is coupled to a controller, such as a control unit 102, for example. In various embodiments, the control unit 102 may be formed integrally with the generator 145 or may be provided as a separate circuit module or device electrically coupled to the generator 145 (shown in phantom to illustrate this option). Although in the presently disclosed embodiment, the generator 145 is shown separate from the to electrosurgical instrument 101, in one embodiment, the generator 145 (and/or the control unit 102) may be formed integrally with the electrosurgical instrument 101 to form a unitary electrosurgical system 100. In various embodiments, the electrosurgical system 100 may also comprise various indicators, such as lights, chimes, buzzers, or meters, for example, to indicate characteristics of the tissue being manipulated by the end effector 110.

The generator 145 may comprise an input device 147 located on a front panel of the generator 145 console. The input device 147 may comprise any suitable device that generates signals suitable for programming the operation of the generator 145, such as a keyboard, or input port, for example. In one embodiment, various electrodes in the first jaw 120A and the second jaw 120B may be coupled to an generator 145 (i.e., an Rf source) and control unit 102. A cable 152 may comprise multiple electrical conductors for the application of electrical energy to positive (+) and negative (−) electrodes of the electrosurgical instrument 101. As discussed in more detail below, the control unit 102 may be used to activate electrical generator 145 and control the delivery of power to the electrodes during operation of the electrosurgical instrument 101.

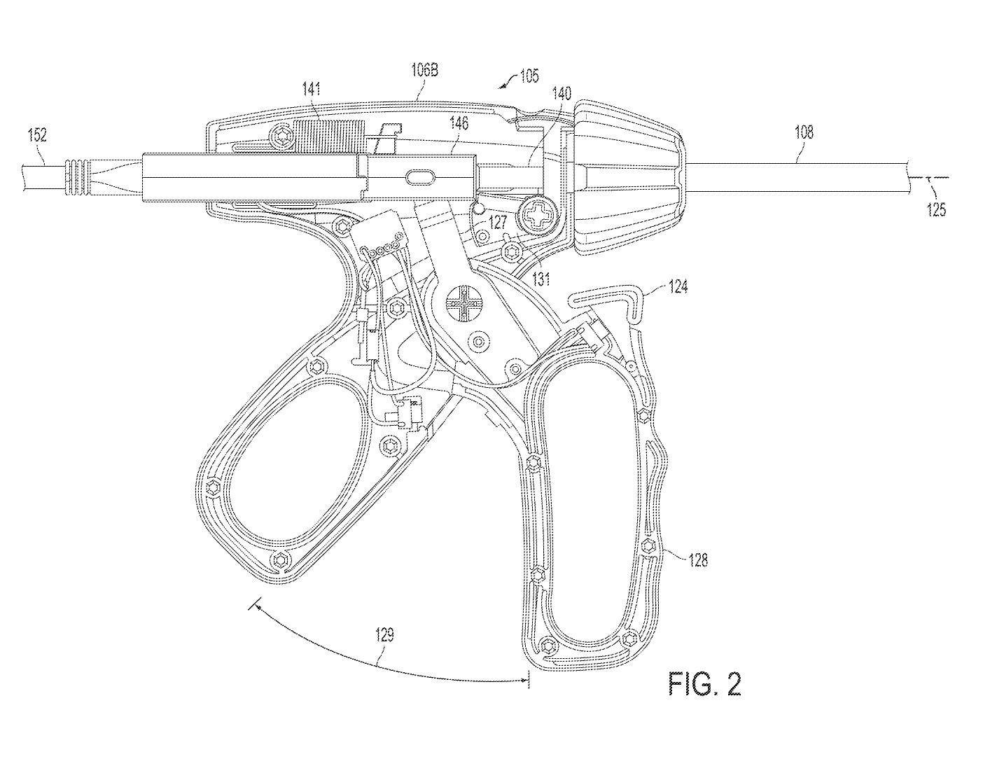

Moving now to FIG. 2, a side view of the handle 105 is shown with half of a first handle body 106A (see FIG. 1) removed to illustrate various components within second handle body 106B. The handle 105 may comprise a lever arm 128 which may be pulled along a path 129. The lever arm 128 may be coupled to a movable cutting member 140 disposed within elongate shaft 108 by a shuttle 146 operably engaged to an extension 127 of lever arm 128. The shuttle 146 may further be connected to a biasing device, such as a spring 141, which may also be connected to the second handle body 106B, to bias the shuttle 146 and thus the cutting member 140 in a proximal direction, thereby urging the jaws 120A and 120B to an open position as seen in FIG. 1. Also, referring to FIGS. 1 and 2, a locking member 131 (see FIG. 2) may be moved by a locking switch 130 (see FIG. 1) between a locked position, where the shuttle 146 is substantially prevented from moving distally as illustrated, and an unlocked position, where the shuttle 146 may be allowed to freely move in the distal direction, toward the elongate shaft 108. The handle 105 can be any type of pistol-grip or other type of handle known in the art that is configured to carry actuator levers, triggers or sliders for actuating the first jaw 120A and the second jaw 120B. The elongate shaft 108 may have a cylindrical or rectangular cross-section, for example, and can comprise a thin-wall tubular sleeve that extends from handle 105. The elongate shaft 108 may include a bore extending therethrough for carrying actuator mechanisms, for example, the cutting member 140, for actuating the jaws and for carrying electrical leads for delivery of electrical energy to electrosurgical components of the end effector 110.

The end effector 110 may be adapted for capturing and transecting tissue and for the contemporaneously welding the captured tissue with controlled application of energy (i.e. Rf energy). The first jaw 120A and the second jaw 120B may close to thereby capture or engage tissue about a longitudinal axis 125 defined by cutting member 140. The first jaw 120A and second jaw 120B may also apply compression to the tissue. In some embodiments, the elongate shaft 108, along with first jaw 120A and second jaw 120B, can be rotated a full 360° degrees, as shown by arrow 117 (FIG. 1), relative to handle 105 through, for example, a rotary triple contact. The first jaw 120A and the second jaw 120B can remain openable and/or closeable while rotated.

FIGS. 3 and 4 illustrate perspective views of the end effector 110 in accordance with one non-limiting embodiment. FIG. 3 shows end the effector 110 in an open configuration and FIG. 4 shows the end effector 110 in a closed configuration. As noted above, the end effector 110 may comprise the upper first jaw 120A and the lower second jaw 120B. Further, the first jaw 120A and second jaw 120B may each have tissue-gripping elements, such as teeth 143, disposed on the inner portions of first jaw 120A and second jaw 120B. The first jaw 120A may comprise an upper first jaw body 161A with an upper first outward-facing surface 162A and an upper first energy delivery surface 175A. The second jaw 120B may comprise a lower second jaw body 161B with a lower second outward-facing surface 162B and a lower second energy delivery surface 175B. The first energy delivery surface 175A and the second energy delivery surface 175B may both extend in a “U” shape about the distal end of the end effector 110.

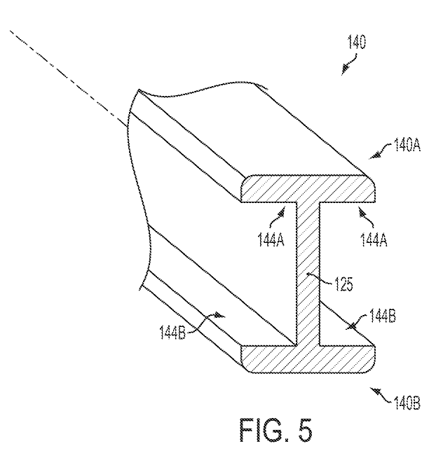

Referring briefly now to FIG. 5, a portion of the cutting member 140 is shown. The lever arm 128 of the handle 105 (FIG. 2) may be adapted to actuate the cutting member 140 which also functions as a jaw-closing mechanism. For example, the cutting member 140 may be urged distally as the lever arm 128 is pulled proximally along the path 129 via the shuttle 146, as shown in FIG. 2 and discussed above. The cutting member 140 may comprise one or several pieces, but in any event, may be movable or translatable with respect to the elongate shaft 108 and/or the jaws 120A, 120B. Also, in at least one embodiment, the cutting member 140 may be made of 17-4 precipitation hardened stainless steel. The distal end of cutting member 140 may comprise a flanged “I”-beam configured to slide within the channels 142A and 142B in jaws 120A and 120B. The cutting member 140 may slide within the channels 142A, 142B to open and close first jaw 120A and second jaw 120B. The distal end of the cutting member 140 may also comprise an upper flange or “c”-shaped portion 140A and a lower flange or “c”-shaped portion 140B. The flanges 140A and 140B respectively define inner cam surfaces 144A and 1448 for engaging outward facing surfaces of first jaw 120A and second jaw 120B. The opening-closing of jaws 120A and 120B can apply very high compressive forces on tissue using cam mechanisms which may include movable “I-beam” cutting member 140 and the outward facing surfaces 162A, 162B of jaws 120A, 120B.

More specifically, referring now to FIGS. 3-5, collectively, the inner cam surfaces 144A and 144B of the distal end of cutting member 140 may be adapted to slidably engage the first outward-facing surface 162A and the second outward-facing surface 162B of the first jaw 120A and the second jaw 120B, respectively. The channel 142A within first jaw 120A and the channel 142B within the second jaw 120B may be sized and configured to accommodate the movement of the cutting member 140, which may comprise a tissue-cutting element, for example, a sharp distal edge. FIG. 4, for example, shows the distal end of the cutting member 140 advanced at least partially through channels 142A and 142B (FIG. 3). The advancement of the cutting member 140 can close the end effector 110 from the open configuration shown in FIG. 3. In the closed position shown by FIG. 4, the upper first jaw 120A and lower second jaw 120B define a gap or dimension D between the first energy delivery surface 175A and second energy delivery surface 175B of first jaw 120A and second jaw 120B, respectively. In various embodiments, dimension D can equal from about 0.0005″ to about 0.040″, for example, and in some embodiments, between about 0.001″ to about 0.010″, for example. Also, the edges of the first energy delivery surface 175A and the second energy delivery surface 175B may be rounded to prevent the dissection of tissue.

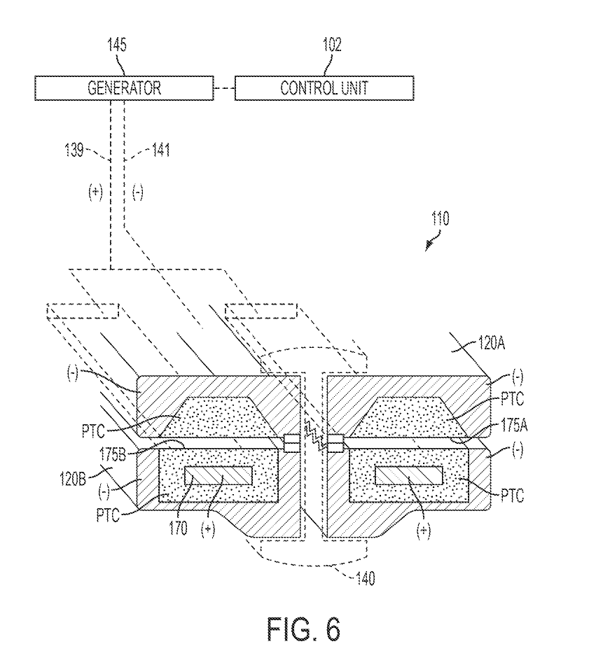

FIG. 6 is a sectional view of the end effector 110 in accordance with one non-limiting embodiment. In one embodiment, the engagement, or tissue-contacting, surface 175B of the lower jaw 120B is adapted to deliver energy to tissue, at least in part, through a conductive-resistive matrix, such as a variable resistive positive temperature coefficient (PTC) body, as discussed in more detail below. At least one of the upper and lower jaws 120A, 120B may carry at least one electrode 170 configured to deliver the energy from the generator 145 to the captured tissue. The engagement, or tissue-contacting, surface 175A of upper jaw 120A may carry a similar conductive-resistive matrix (i.e., a PTC material), or in some embodiments the surface may be a conductive electrode or an insulative layer, for example. Alternatively, the engagement surfaces of the jaws can carry any of the energy delivery components disclosed in U.S. Pat. No. 6,773,409, filed Oct. 22, 2001, entitled “ELECTROSURGICAL JAW STRUCTURE FOR CONTROLLED ENERGY DELIVERY,” which is incorporated herein by reference.

The first energy delivery surface 175A and the second energy delivery surface 175B may each be in electrical communication with the generator 145. The first energy delivery surface 175A and the second energy delivery surface 175B may be configured to contact tissue and deliver electrosurgical energy to captured tissue which are adapted to seal or weld the tissue. The control unit 102 regulates the electrical energy delivered by electrical generator 145 which in turn delivers electrosurgical energy to the first energy delivery surface 175A and the second energy delivery surface 175B. The energy delivery may be initiated by an activation button 124 (FIG. 2) operably engaged with the lever arm 128 and in electrical communication with the generator 145 via cable 152. In one embodiment, the electrosurgical instrument 101 may be energized by the generator 145 by way of a foot switch 144 (FIG. 1). When actuated, the foot switch 144 triggers the generator 145 to deliver electrical energy to the end effector 110, for example. The control unit 102 may regulate the power generated by the generator 145 during activation. Although the foot switch 144 may be suitable in many circumstances, other suitable types of switches can be used.

In various embodiments, as shown in FIGS. 1 and 6, the electrosurgical system 100 may comprise at least one supply conductor 139 and at least one return conductor 141, wherein current can be supplied to electrosurgical instrument 101 via the supply conductor 139 and wherein the current can flow back to the generator 145 via return conductor 141. In various embodiments, the supply conductor 139 and the return conductor 141 may comprise insulated wires and/or any other suitable type of conductor. In certain embodiments, as described below, the supply conductor 139 and the return conductor 141 may be contained within and/or may comprise the cable 152 extending between, or at least partially between, the generator 145 and the end effector 110 of the electrosurgical instrument 101. In any event, the generator 145 can be configured to apply a sufficient voltage differential between the supply conductor 139 and the return conductor 141 such that sufficient current can be supplied to the end effector 110.

As mentioned above, the electrosurgical energy delivered by electrical generator 145 and regulated, or otherwise controlled, by the control unit 102 may comprise radio frequency (Rf) energy, or other suitable forms of electrical energy. Further, the opposing first and second energy delivery surfaces 175A and 175B may carry variable resistive positive temperature coefficient (PTC) bodies that are in electrical communication with the generator 145 and the control unit 102. In some embodiments, the end effector may also carry a positive temperature heat conduction (PTHC) material to assist in dissipating heat. When the PTHC material is relatively cool, it generally does not conduct heat away from the end effector. The PTHC material increases the amount of heat it dissipates once the temperature of the end effector is relatively high and a tissue seal has been made. Selectively dissipating with the PTHC material the heat helps to reduce burning or overheating of tissue. For example, heat can be maintained at the tissue until proper temperatures for sealing have been reached, and then the PTHC material dissipates excess heat away from the area to help reduce overheating. Additional details regarding electrosurgical end effectors, jaw closing mechanisms, and electrosurgical energy-delivery surfaces are described in the following U.S. patents and published patent applications: U.S. Pat. Nos. 7,087,054; 7,083,619; 7,070,597; 7,041,102; 7,011,657; 6,929,644; 6,926,716; 6,913,579; 6,905,497; 6,802,843; 6,770,072; 6,656,177; 6,533,784; and 6,500,176; and U.S. Pat. App. Pub. Nos. 2010/0036370 and 2009/0076506, all of which are incorporated herein in their entirety by reference and made a part of this specification.

In one embodiment, the generator 145 may be implemented as an electrosurgery unit (ESU) capable of supplying power sufficient to perform bipolar electrosurgery using radio frequency (Rf) energy. In one embodiment, the ESU can be a bipolar ERBE ICC 350 sold by ERBE USA, Inc. of Marietta, Ga. In some embodiments, such as for bipolar electrosurgery applications, a surgical instrument having an active electrode and a return electrode can be utilized, wherein the active electrode and the return electrode can be positioned against, adjacent to and/or in electrical communication with, the tissue to be treated such that current can flow from the active electrode, through the positive temperature coefficient (PTC) bodies and to the return electrode through the tissue. Thus, in various embodiments, the electrosurgical system 100 may comprise a supply path and a return path, wherein the captured tissue being treated completes, or closes, the circuit.

In various embodiments, the electrosurgical system 100 may comprise various indicators to inform the user about the energy delivery levels and/or the status of the tissue. In some embodiments, the indicators may provide information related to tissue impedance or tissue temperature, for example.

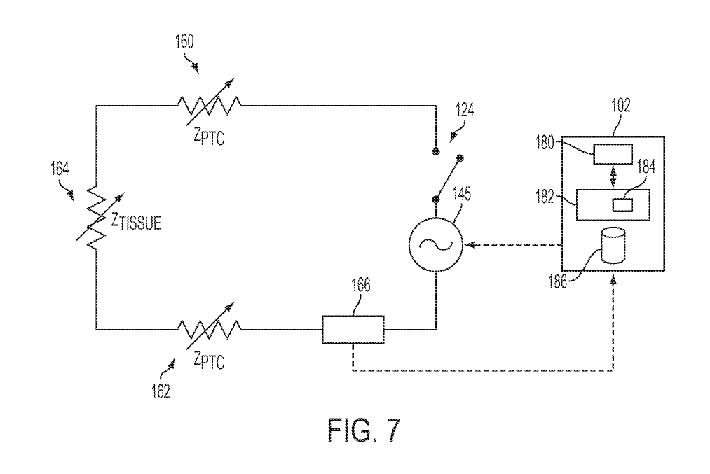

FIG. 7 illustrates a simplified circuit diagram of the end effector 110 during use. The generator 145 supplies energy to a circuit comprising elements having variable impedances, such as PTC elements 160, 162, and the captured tissue 164. Various characteristics of the circuit may be measured during use. Based on the measurements, the energy delivered to the electrosurgical instrument 101 may be varied accordingly. For example, the circuit's current may be measured through any suitable measurement or sensing technique. The current through the captured tissue may be measured by way of a return electrode provided on one of the first jaw 120A and the second jaw 120B. In one embodiment, a current sensing circuit 166 comprising a current sensing resistor may be positioned in series with the return electrode. The current sensing circuit 166 may provide data to the control unit 102. The data is analyzed by the control unit 102, and the control unit 102 regulates the power delivered to the tissue by the generator 145. In some embodiments, hall effect current sensing transducers may be used to provide data regarding the current flowing through captured tissue. In some embodiments, magnetoresistive field sensors may be used to provide data regarding the current flowing through captured tissue. Other embodiments may utilize current sensing circuitry using other current measuring and/or impedance measuring techniques. All such embodiment are intended to be within the scope of the present disclosure.

The control unit 102 may also comprise, or otherwise be in communication with a processor 180. The control unit 102 may also comprise a computer memory 182 in communication with the processor 180. Software with instructions 184 for execution by the processor 180 may be stored on the computer memory 182. The processor 180 may execute the software to perform various functions, such as perform control the power delivered to captured tissue, as disclosed herein. The control unit 102 may also comprise a storage structure 186 for storing data, such as PTC impedance data, and/or any other types of data useful in controlling the power delivered by the generator 145. The control unit 102 may comprise one or more processors 180 and one or more computer memories 182. For convenience, only one processor 180 and only one memory 182 are shown in FIG. 7. The processor 180 may be implemented as an integrated circuit (IC) having one or multiple cores. The memory 182 may comprise volatile and/or non-volatile memory units. Volatile memory units may comprise random access memory (RAM), for example. Non-volatile memory units may comprise read only memory (ROM), for example, as well as mechanical non-volatile memory systems, such as, for example, a hard disk drive, an optical disk drive, etc. The RAM and/or ROM memory units may be implemented as discrete memory ICs, for example.

Through measurement of the circuit's current, both the overall electrical impedance (Z) of the circuit and the rate of change of the impedance (dZ/dt) may be determined based on the relationship of voltage to current (i.e., Ohms' law). The impedance and rate of change of the impedance for the tissue component of the circuit may then be determined, or at least approximated by the control unit 102, based on EQs. 1 and 2.

$$\begin{matrix}{Z}_{TOTAL} = {Z}_{TISSUE} + {Z}_{PTC} + {Z}_{GENERATOR} & EQ . 1 \\ \frac{d {Z}_{TOTAL}}{d t} = \frac{d {Z}_{TISSUE}}{d t} + \frac{d {Z}_{PTC}}{d t} + \frac{d {Z}_{GENERATOR}}{d t} & EQ . 2\end{matrix}$$

The impedance of the generator may be approximately zero (or in some cases is a known value.) Similarly, the impedance of the PTC material for various current levels may be calibrated and considered a known value. Accordingly, EQs. 1 and 2 may be solved for the tissue component to yield EQs. 3 and 4.

$$\begin{matrix}{Z}_{TISSUE} = {Z}_{PTC} - {Z}_{TOTAL} & EQ . 3 \\ \frac{d {Z}_{TISSUE}}{d t} = \frac{d {Z}_{TOTAL}}{d t} - \frac{d {Z}_{PTC}}{d t} & EQ . 4\end{matrix}$$

The power delivered to the circuit by the generator may be determined by the control unit 102 as a function of both the impedance of the tissue and the rate of change of the impedance of the tissue, as indicated in EQ. 5.

$$POWER = P ( {Z}_{TISSUE} , \frac{d {Z}_{TISSUE}}{d t} ) \qquad EQ . 5$$

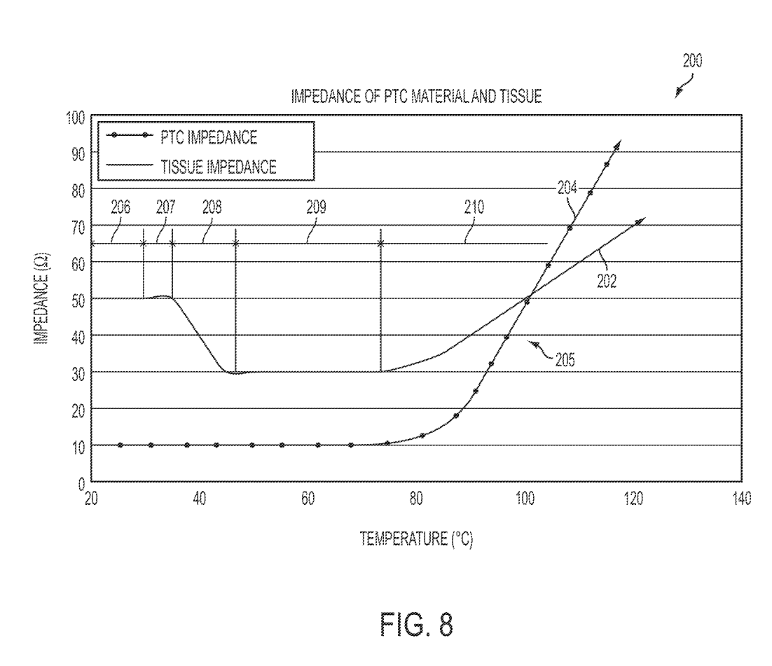

FIG. 8 is a graph 200 of a tissue impedance curve 202 and a PTC material impedance curve 204 as a function of temperature in accordance with one non-limiting embodiment. As illustrated, the impedance of the PTC material is relatively low until a the material has reached a certain temperature range. For example, the impedance of the PTC starts to increase sharply starting around 85° C. As described previously, the PTC material may be positioned intermediate tissue and the electrode during use of the instrument. In some embodiments, the PTC material contacts the captured tissue. In any event, the PTC material serves to reduce current flow through the tissue when the temperature of the tissue increases, thereby decreasing the likelihood of overheating the tissue.

Still referring to FIG. 8, the tissue impedance curve 202 indicates that the impedance of tissue varies based on the temperature of the tissue. Additionally, the rate of change of the impedance (i.e., the “slope” of the change) varies with temperature as well. For example, in one example tissue, the tissue's impedance is about 50 ohms at 20° C., shown as zone 206. When the tissue is heated, a slight increase in impedance occurs, shown as zone 207, followed by a relatively steep decay, shown as zone 208. Eventually, the impedance stabilizes (zone 209) and after increasing to about 75° C., the impedance of the tissue slowly rises to its original impedance (i.e., 50 ohms) and then continues to rise as the tissue heats and cauterizes (zone 210). Accordingly, the tissue temperature may be estimated based on a combination of the impedance and/or current levels and the rate of the change of the impedance and/or current level. Since the tissue temperature may be ascertained, the energy delivered to the tissue may be controlled to reduce the unwanted effects of overheating the tissue during a welding procedure.

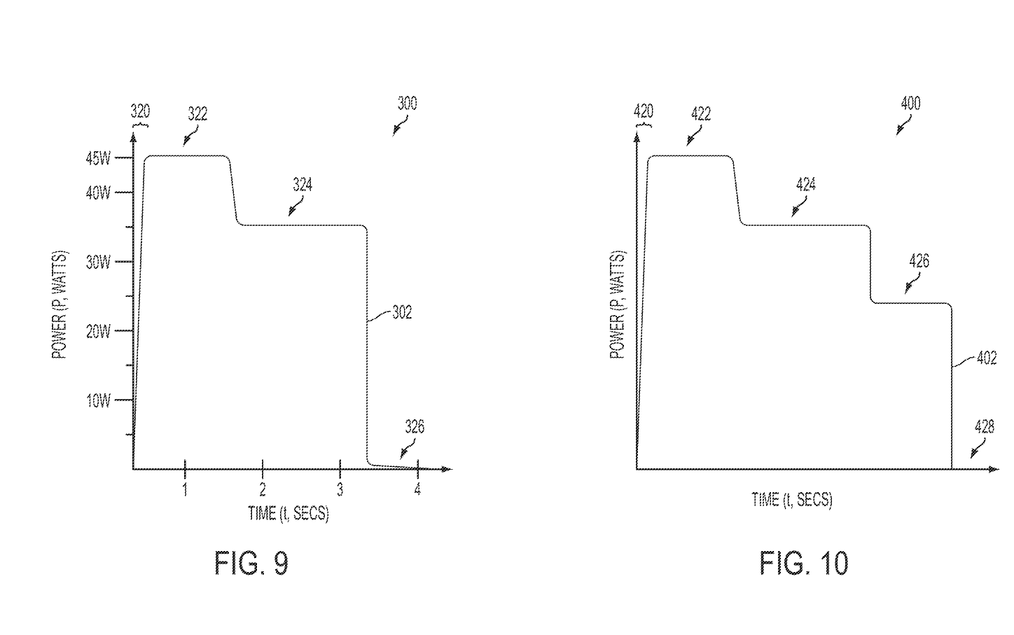

FIG. 9 is a graph 300 of the power level curve 302 controlled by the control unit 102 and the power delivered to the circuit by the generator 145 over time in accordance with one non-limiting embodiment. The overall shape of the curve is determined by both the tissue impedance and the rate of change of the tissue impedance. Generally, the power level curve 302 allows for a faster cut than conventional power level curves. Further, when a slow cut is used, the power level curve 302 reduces the likelihood of the tissue becoming overheated or burned. As is to be appreciated, the power level curve 302 may be generated through any suitable technique, such as pulse width modulation (PWM) for example. In one embodiment, the control unit 102 is configured to deliver pulses of energy to the circuit and vary the level of energy by varying a duty cycle of the pulses of energy. Further, the control unit 102 is configured to vary the level of energy by varying an amplitude of a pulse of energy. Accordingly, any suitable power generation technique may be used to generate the power level curve 302.

In the illustrated embodiment, the power level curve has four general zones or sections 320, 322, 324, and 326. The first zone 320 is a relatively steep section of the curve which quickly ramps up the power delivered to the tissue. The second zone 322 is a section of sustained energy delivery at a relatively high level. In one embodiment, the power of zone 322 is approximately 45 Watts. This power level may be adjusted based on the type of tissue or the thickness of tissue, for example. In order to reduce the amount of energy delivered to the tissue, the power delivered to the circuit may by stepped down to the level indicated at the zone 324. After sufficient energy has been delivered to the tissue to effectively seal the tissue, the power delivered to the tissue may be reduced to zero (zone 326).

During the application of the energy to the tissue, the electrosurgical system 100 monitors the current flow through the tissue and the rate of change of the current flow through the tissue. Through the monitoring of the current flow, the impedance may also be calculated and monitored. The amount of energy, and the length of the time the energy is delivered to captured tissue, will vary based on the tissue impedance and the rate of change of the tissue impedance. Thus, in one embodiment, 45 W of power may be delivered to the tissue for approximately 1.8 seconds, as illustrated by zone 322 in FIG. 9. In another embodiment, for example, the 45 W of power may be delivered for more or less time, depending on the amount of current flowing through the tissue and the rate of change of the current flowing through the tissue. Thus, the temporal durations of the various levels in power illustrated in FIG. 9 are determined by the control unit 102 as a function the captured tissue's impedance and the rate of the change of the impedance. The temporal durations of the various zones in FIG. 9 are merely in accordance with one embodiment.

Referring now to FIGS. 8-9, upon activation by the activation button 124 (FIG. 2), the power level delivered to the tissue is quickly ramped (FIG. 9, zone 320) and then a sustained level of power delivery is maintained (FIG. 9, zone 322). In reaction to this surge of energy, the tissue will heat and first experience a slight rise in impedance (FIG. 8, zone 207), followed by a sharp drop in impedance (FIG. 8, zone 208). As the sustained level of power is continually delivered to the tissue, the tissue will continue to heat and experience a generally sustained impedance level (FIG. 8, zone 209). Eventually, the tissue will experience a gradual rise in impedance (FIG. 8, zone 210).

As the tissue progresses through these various impedance changes, the electrosurgical system 100 will detect the impedance level as well as the rate of change of the impedance and supply the information to the control unit. In various embodiments, the generator 145 may continue to deliver a relatively high level of power (FIG. 9, zone 322) until the tissue impedance increases at a relatively low rate (FIG. 8, zone 210). The detection of the tissue impedance increasing at a relatively low rate indicates that the tissue is starting to reach sufficient temperature to properly cauterize. In order to reduce the amount of energy delivered to the tissue, the power delivered to the circuit may by stepped down to the level indicated at the zone 324 (FIG. 9) by the control unit 102. After sufficient energy has been delivered to the tissue to effectively seal the tissue, the power delivered to the tissue may be reduced to zero by the control unit 102, as indicated at zone 326 (FIG. 9).

FIG. 10 is a graph 400 of the power level curve 402 delivered to the circuit by the generator 145 over time in accordance with another non-limiting embodiment. As illustrated, the power level curve 402 has five general zones or sections 420, 422, 424, 426, and 428. The first zone 420 is relatively steep section of the curve which quickly ramps up the power delivered to the tissue. The second zone 422 is a section of sustained energy delivery at a relatively high level. In some embodiments, the power delivery of zone 422 may be greater and less than 45 W. In order to reduce the amount of energy delivered to the tissue, the power delivered to the circuit may by stepped down to the level indicated at the zone 424. In order to further reduce the amount of energy delivered to the tissue, the power delivered to the circuit may by stepped down to the level indicated at the zone 426. After sufficient energy has been delivered to the tissue to effectively seal the tissue, the power delivered to the tissue may be reduced to zero (zone 428). In this embodiment, for example, the power delivered to the captured tissue when the tissue impedance curve is in zone 210 (FIG. 8) has multiple steps (e.g., FIG. 10, zones 424 and 426). Furthermore, while various zones in FIGS. 9 and 10 are illustrated as applying a single power level for a certain duration, in some embodiments, the power levels within the discrete zones may increase and/or decrease. Thus, as illustrated in FIGS. 9 and 10, the power curve provided by the generator 145 to the tissue is not limited to a particular shape or output.

The embodiments of the devices described herein may be introduced inside a patient using minimally invasive or open surgical techniques. In some instances it may be advantageous to introduce the devices inside the patient using a combination of minimally invasive and open surgical techniques. Minimally invasive techniques may provide more accurate and effective access to the treatment region for diagnostic and treatment procedures. To reach internal treatment regions within the patient, the devices described herein may be inserted through natural openings of the body such as the mouth, anus, and/or vagina, for example. Minimally invasive procedures performed by the introduction of various medical devices into the patient through a natural opening of the patient are known in the art as NOTES™ procedures. Some portions of the devices may be introduced to the tissue treatment region percutaneously or through small-keyhole-incisions.

Endoscopic minimally invasive surgical and diagnostic medical procedures are used to evaluate and treat internal organs by inserting a small tube into the body. The endoscope may have a rigid or a flexible tube. A flexible endoscope may be introduced either through a natural body opening (e.g., mouth, anus, and/or vagina) or via a trocar through a relatively small-keyhole-incision incisions (usually 0.5-1.5 cm). The endoscope can be used to observe surface conditions of internal organs, including abnormal or diseased tissue such as lesions and other surface conditions and capture images for visual inspection and photography. The endoscope may be adapted and configured with working channels for introducing medical instruments to the treatment region for taking biopsies, retrieving foreign objects, and/or performing surgical procedures.

The devices disclosed herein may be designed to be disposed of after a single use, or they may be designed to be used multiple times. In either case, however, the device may be reconditioned for reuse after at least one use. Reconditioning may include a combination of the steps of disassembly of the device, followed by cleaning or replacement of particular pieces, and subsequent reassembly. In particular, the device may be disassembled, and any number of particular pieces or parts of the device may be selectively replaced or removed in any combination. Upon cleaning and/or replacement of particular parts, the device may be reassembled for subsequent use either at a reconditioning facility, or by a surgical team immediately prior to a surgical procedure. Those of ordinary skill in the art will appreciate that the reconditioning of a device may utilize a variety of different techniques for disassembly, cleaning/replacement, and reassembly. Use of such techniques, and the resulting reconditioned device, are all within the scope of this application.

Preferably, the various embodiments of the devices described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility. Other sterilization techniques can be done by any number of ways known to those skilled in the art including beta or gamma radiation, ethylene oxide, and/or steam.

Although the various embodiments of the devices have been described herein in connection with certain disclosed embodiments, many modifications and variations to those embodiments may be implemented. For example, different types of end effectors may be employed. Also, where materials are disclosed for certain components, other materials may be used. The foregoing description and following claims are intended to cover all such modification and variations.

In various embodiments, modules or software can be used to practice certain aspects of the invention. For example, software-as-a-service (SaaS) models or application service provider (ASP) models may be employed as software application delivery models to communicate software applications to clients or other users. Such software applications can be downloaded through an Internet connection, for example, and operated either independently (e.g., downloaded to a laptop or desktop computer system) or through a third-party service provider (e.g., accessed through a third-party web site). In addition, cloud computing techniques may be employed in connection with various embodiments of the invention.

Moreover, the processes associated with the present embodiments may be executed by programmable equipment, such as computers, or other processor-based devices. Software or other sets of instructions that may be employed to cause programmable equipment to execute the processes may be stored in any storage device, such as, for example, a computer system (non-volatile) memory, an optical disk, magnetic tape, or magnetic disk. Furthermore, some of the processes may be programmed when the computer system is manufactured or via a computer-readable memory medium.

It can also be appreciated that certain process aspects described herein may be performed using instructions stored on a computer-readable memory medium or media that direct a computer or computer system to perform process steps. A computer-readable medium may include, for example, memory devices such as diskettes, compact discs of both read-only and read/write varieties, optical disk drives, and hard disk drives. A computer-readable medium may also include memory storage that may be physical, virtual, permanent, temporary, semi-permanent and/or semi-temporary.

A “computer,” “computer system,” “host,” “engine,” or “processor” may be, for example and without limitation, a processor, microcomputer, minicomputer, server, mainframe, laptop, personal data assistant (PDA), wireless e-mail device, cellular phone, pager, processor, fax machine, scanner, or any other programmable device configured to transmit and/or receive data over a network. Computer systems and computer-based devices disclosed herein may include memory for storing certain software applications used in obtaining, processing, and communicating information. It can be appreciated that such memory may be internal or external with respect to operation of the disclosed embodiments. The memory may also include any means for storing software, including a hard disk, an optical disk, floppy disk, ROM (read only memory), RAM (random access memory), PROM (programmable ROM), EEPROM (electrically erasable PROM) and/or other computer-readable memory media.

In various embodiments of the present invention, a single component may be replaced by multiple components, and multiple components may be replaced by a single component, to perform a given function or functions. Except where such substitution would not be operative to practice embodiments of the present invention, such substitution is within the scope of the present invention.

In general, it will be apparent to one of ordinary skill in the art that various embodiments described herein, or components or parts thereof, may be implemented in many different embodiments of software, firmware, and/or hardware, or modules thereof. The software code or specialized control hardware used to implement some of the present embodiments is not limiting of the present invention. For example, the embodiments described hereinabove may be implemented in computer software using any suitable computer programming language such as .NET, SQL, MySQL, or HTML using, for example, conventional or object-oriented techniques. Programming languages for computer software and other computer-implemented instructions may be translated into machine language by a compiler or an assembler before execution and/or may be translated directly at run time by an interpreter. Examples of assembly languages include ARM, MIPS, and x86; examples of high level languages include Ada, BASIC, C, C++, C#, COBOL, Fortran, Java, Lisp, Pascal, Object Pascal; and examples of scripting languages include Bourne script, JavaScript, Python, Ruby, PHP, and Perl. Such software may be stored on any type of suitable computer-readable medium or media such as, for example, a magnetic or optical storage medium. Thus, the operation and behavior of the embodiments are described without specific reference to the actual software code or specialized hardware components. The absence of such specific references is feasible because it is clearly understood that artisans of ordinary skill would be able to design software and control hardware to implement the embodiments of the present invention based on the description herein with only a reasonable effort and without undue experimentation.

In various embodiments, computers and computer systems described herein may have the following main components: arithmetic and logic unit (ALU), control unit, memory, and input and output devices (I/O devices). These components can be interconnected by busses, often comprising groups of wires or cables. The control unit, ALU, registers, and basic I/O (and often other hardware closely linked with these sections) can be collectively considered a central processing unit (CPU) for the computer system. The CPU may be constructed on a single integrated circuit or microprocessor.

The control unit (control system or central controller) directs the various components of a computer system. The control system decodes each instruction in a computer program and turns it into a series of control signals that operate other components of the computer system. To enhance performance or efficiency of operation, the control system may alter the order of instructions. One component of the control unit is the program counter, a memory register that tracks the location in memory from which the next instruction is to be read.

The ALU is capable of performing arithmetic and logic operations. The set of arithmetic operations that a particular ALU supports may be limited to adding and subtracting or might include multiplying or dividing, trigonometry functions (sine, cosine, etc.) and square roots. Some may be programmed to operate on whole numbers (integers), while others use floating point to represent real numbers, for example. An ALU may also compare numbers and return Boolean truth values (e.g., true or false). Superscalar computers may contain multiple ALUs to facilitate processing multiple instructions at the same time. For example, graphics processors and computers with SIMD and MIMD features often possess ALUs that can perform arithmetic operations on vectors and matrices. Certain computer systems may include one or more RAM cache memories configured to move more frequently needed data into the cache automatically.

Examples of peripherals that may be used in connection with certain embodiments of the invention include input/output devices such as keyboards, mice, screen displays, monitors, printers, hard disk drives, floppy disk drives, joysticks, and image scanners.

Embodiments of the methods and systems described herein may divide functions between separate CPUs, creating a multiprocessing configuration. For example, multiprocessor and multi-core (multiple CPUs on a single integrated circuit) computer systems with co-processing capabilities may be employed. Also, multitasking may be employed as a computer processing technique to handle simultaneous execution of multiple computer programs.

In various embodiments, the systems and methods described herein may be configured and/or programmed to include one or more of the above-described electronic, computer-based elements and components. In addition, these elements and components may be particularly configured to execute the various rules, algorithms, programs, processes, and method steps described herein.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. An electrosurgical system, comprising:
a control unit comprising a processor and memory in communication with the processor, wherein the control unit is configured to deliver a variable level of energy to a circuit comprising an electrode and tissue in electrical communication with the electrode, wherein the control unit is configured to vary the level of energy delivered to the tissue based on:
a current flowing through the tissue; and
a rate of change of the current flowing through the tissue.

2. The electrosurgical system of claim 1, wherein the energy is radio frequency (Rf) energy.

3. The electrosurgical system of claim 1, wherein the control unit is configured to deliver pulses of energy to the circuit and vary the level of energy by varying a duty cycle of the pulses of energy.

4. The electrosurgical system of claim 3, wherein the control unit is configured to vary the level of energy by varying an amplitude of a pulse of energy.

5. The electrosurgical system of claim 1, wherein the control unit is configured to reduce the level of energy delivered to the circuit when the current flowing through the tissue reaches a predetermined current level and the rate of change of the current flowing through the tissue reaches a predetermined rate of change.

6. The electrosurgical system of claim 1, wherein the control unit configured to deliver the variable level of energy during a time interval comprising at least a first portion of time preceding a second portion of time, wherein a first level of energy is delivered during the first portion and a second level of energy is delivered during the section portion, and wherein the first level is higher than the second level.

7. The electrosurgical system of claim 1, comprising a:
a handle;
an elongate shaft extending distally from the handle;
a trigger operably connected to the elongate shaft; and
an end effector coupled to the distal end of the elongate shaft, wherein the end effector comprises:
a first jaw member;
a second jaw member, wherein the first jaw member is moveable relative to the second jaw member between an open and a closed position;
an axially movable member configured to open and close the jaws; and
the electrode in electrical communication with the control unit.

8. The electrosurgical system of claim 1, further comprising a positive temperature coefficient (PTC) coupled to one of the first and second jaw members.

9. An electrosurgical system, comprising:
a control unit configured to deliver a variable level of radio frequency (Rf) energy to a circuit comprising an electrode and tissue in electrical communication with the electrode, wherein the control unit is configured to vary the level of Rf energy delivered to the tissue based on:
an impedance of the tissue; and
a rate of change of the impedance of the tissue;
a handle;
an elongate shaft extending distally from the handle;
a trigger operably connected to the elongate shaft;
an end effector coupled to the distal end of the elongate shaft, wherein the end effector comprises:
a first jaw member;
a second jaw member, wherein the first jaw member is moveable relative to the second jaw member between an open and a closed position; and
an axially movable cutting member.

10. The electrosurgical system of claim 9, wherein the control unit is configured to reduce the level of Rf energy delivered to the circuit when the impedance of the tissue reaches a predetermined current level and the rate of change of the impedance of the tissue reaches a predetermined rate of change.

11. The electrosurgical system of claim 9, wherein the end effector comprises a positive temperature coefficient (PTC) material coupled to at least one of the first and second jaw members.

12. The electrosurgical system of claim 9, further comprising an radio frequency (Rf) generator.

13. The electrosurgical system of claim 9, wherein the end effector comprises a positive temperature heat conduction material.

14. The electrosurgical system of claim 9, wherein the control unit is configured to deliver pulses of energy to the circuit and vary the level of energy by varying a duty cycle of the pulses of energy, and wherein the control unit is configured to vary the level of energy by varying an amplitude of a pulse of energy.

15. An apparatus, comprising:
a radio frequency (Rf) energy delivery source configured to electrically couple to a circuit comprising an electrode and tissue, the energy delivery source comprising:
a current sensing circuit, configured to sense the current through the tissue during the delivery of the energy; and
a controller configured to receive a signal from the current sensing circuit and vary an output level of the energy based on a level of an impedance of the tissue and a rate of change of the impedance of the tissue.

16. The apparatus of claim 15, wherein the current sensing circuit comprises a current sensing resistor in electrical communication with the electrode.

17. The apparatus of claim 16, wherein the controller is configured to the level of energy delivered to the circuit when the current flowing through the tissue reaches a first current level and the rate of change of the current flowing through the tissue reaches a first rate of change.

18. The apparatus of claim 17, wherein the controller is configured to adjust the level of energy delivered to the circuit when the current flowing through the tissue reaches a second current level and the rate of change of the current flowing through the tissue reaches a second rate of change.

19. The apparatus of claim 16, comprising a:
a handle;
an elongate shaft extending distally from the handle;
a trigger operably connected to the elongate shaft;
an end effector coupled to the distal end of the elongate shaft, wherein the end effector comprises:
a first jaw member;
a second jaw member, wherein the first jaw member is moveable relative to the second jaw member between an open and a closed position; and
the electrode in electrical communication with the control unit.

20. The apparatus of claim 19, wherein the end effector comprises a positive temperature coefficient (PTC) material coupled to at least one of the first and second jaw members.
